(19) United States
(12) Patent Application Publication
(10) Pub. No.: US 2026/0053486 A1
(43) Pub. Date: Feb. 26, 2026

(54) RETRACTOR SYSTEM FOR SPINE SURGERY

(71) Applicant: NEO MEDICAL SA, Villette (CH)

(72) Inventors: Lionel SIMON, Solaize (FR); Morten BEYER, Rødkærsbro (DK); Vincent LEFAUCONNIER, Brent (CH)

(21) Appl. No.: 19/104,837

(22) PCT Filed: Sep. 21, 2023
(86) PCT No.: PCT/IB2023/059342

§ 371 Date: Feb. 19, 2025

(30) Foreign Application Priority Data
Oct. 7, 2022 (WO) PCT/IB2022/059604

(51) Int. Cl. A61B 17/02; A61B 90/00; A61F 2/46

(52) U.S. Cl. CPC A61B 17/025; A61B 17/0206; A61B 90/39; A61F 2/4611; A61B 2017/0256; A61B 2090/3966

(57) ABSTRACT

Retractor system for spine surgery comprising two retractor members (1,2) being each made of a blade (1) and a proximal part forming a frame (2), at least two pins (10) having each a distal end configured to be anchored in a vertebra, and a blocking mechanism (5) for maintaining the retractor members (1,2) at a certain distance from each other; wherein at least one retractor member (1,2) contains a channel (3) that is configured to slidably hold said pin (10); characterized by the fact that the channel (3) is oriented along a direction that differs from the blade (1) direction. The invention also relates to a retractor system that includes at least one shim (12) with guiding grooves (15) ending before the shim distal end.

### FIELD OF INVENTION

The present invention generally relates to surgical retractors systems that are configured for approaching the spine with a lateral, anterior or posterior surgical technique.

### BACKGROUND OF THE INVENTION

Spinal surgical retractors systems are used for retracting and maintaining soft tissues away from the surgical site to enable the surgeon to access intervertebral discs (or any other structure of the spine), for instance for removing them partially and place implants such as a cage or screws

A retractor system usually includes at least two blades having each a proximal part forming a frame, two pins having each a distal end configured to be anchored in a vertebra and a blocking mechanism for maintaining the blades at a certain distance from each other.

Retractor systems as defined above are disclosed for instance in patent documents U.S. Pat. Nos. 9,220,491 B2 and 10,470,753 B2.

In some systems each blade contains a channel that is configured to slidably hold one pin. See for instance U.S. Pat. Nos. 8,852,089 B2 and 11,426,152 B2.

In those prior art systems, the channel is oriented along a direction that is parallel to the blade direction.

Shims may also be placed between the blade edges or fixed to a blade to prevent the ingress of unwanted or sensitive biological structures, such as tissues, nerves, or vessels, into the surgical target site, as well as to prevent instruments from passing outside the surgical target site and contacting surrounding tissues or structures. Such shims are disclosed in U.S. Pat. Nos. 9,220,491 B2, 10,470,753 B2 or 8,821,396 B1.

Several disadvantages are associated with prior art systems, in particular the risk of an accidental withdrawal of the blades from the surgical site, due to the pressure imparted on the blades by the surrounding tissues. In addition, the way to expand usually from the frame structure makes it necessary to have very stiff material and a potentially bulky structure.

It is also complicated, if not impossible, to maintain the blades around the surgical site and only retrieve the shims, for instance for allowing an easier access to the surgical site.

### SUMMARY OF THE INVENTION

According to the present invention, several solutions are provided to overcome the drawbacks of the prior art.

To this effect the invention provides a retractor system for spine surgery comprising two retractor members being each made of a blade and a proximal part forming a frame, at least two pins having each a distal end configured to be anchored in a vertebra, and a blocking mechanism for maintaining the blades at a certain distance from each other; wherein at least one retractor member contains a channel that is configured to slidably hold the pin; characterized by the fact that the channel is oriented along a direction that differs from the blade direction.

Preferred embodiments of the invention are defined in the claims.

### BRIEF DESCRIPTION OF THE DRAWINGS

Other features, aspects and advantages of the present invention will be more fully understood when considered with respect to the following detailed description, appended claims, and accompanying drawings where:

FIG. 1 represents an example of a retractor member according to the invention

FIG. 2 represents shows a pair of retractor members as illustrated in FIG. 1

FIG. 3 shows an example of a dilator member that may be used with the retractor members of FIGS. 1 and 2

FIG. 4 shows a dilator made of two dilator members as illustrated on FIG. 3

FIG. 5 shows the dilator of FIG. 4 viewed from above

FIG. 6 shows the same dilator surrounded by two retractor members (top view)

FIG. 7 is a side view of a retractor with a dilator and a pin

FIG. 8 shows a pin

FIG. 9 is like FIG. 7 but shows the angle between the pin and the related blade

FIG. 10 is a schematic representation of the forces acting on the blade and the pin

FIG. 11 is a top view in the blade axis

FIG. 12 is a similar view as in FIG. 11 but in the pin axis

FIG. 13 shows a retractor member with its retention ring

FIG. 14 is a perspective view of a retractor with shims

FIG. 15 represents a shim

FIG. 16 shows a top view of a retractor with shims fixed to the blades

FIG. 17 shows the distal part of a retractor with pins and a shim

FIG. 18 shows the distal part of a shim

FIG. 19 shows a similar configuration as in FIG. 17 but with a shim in a lower position

FIG. 20 shows a shim in two different positions

FIG. 21 represents a top view of a retractor system with respect to a spine portion

FIG. 22 shows a similar configuration as in FIG. 21 but in a perspective view

FIG. 23 shows another example of a retractor according to the invention (perspective top view)

FIG. 24 shows the same object as in FIG. 23, but in a perspective bottom view.

FIG. 25 shows the same object as in FIG. 23, but in a top view.

### Numerical References Used in the Figures

1. Blade
2. Frame
3. Blade groove
4. Arm
5. Ratcheting system
6. Dilator member
7. K-wire channel
8. Guide
9. Dilator groove
10. Pin
11. Retention ring
12. Shim
13. Shim distal end
14. Guiding stop
15. Linear guiding groove

### DETAILED DESCRIPTION OF THE INVENTION

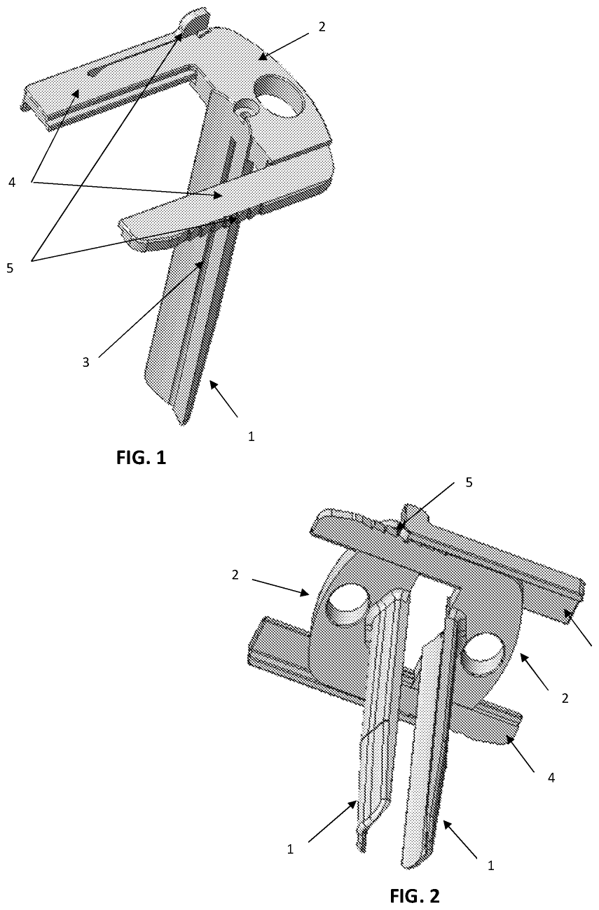

The retractor member 1,2 shown on FIG. 1 is the main one. It is also named cranio-caudal due to its location during the operation. It is made of a blade 1 and a proximal part forming a frame 2 with two arms 4 being each located at a different height.

FIG. 2 shows the retractor basis which is made of the retractor member 1,2 of FIG. 1 and a second retractor member 1,2. The two frames 2 have a complementary shape, in such a way that when both retractor members 1,2 are joined, one arm 4 of the first frame 2 is located above one arm 4 of the second frame 2 and the other arm 4 of the first frame is located below the other arm 4 of the second frame 2.

Each frame 2 includes a ratcheting system 5 for holding the two retractor members 1,2 at a certain distance from each other.

Each blade 1 furthermore include a groove 3 that extends along the full blade length.

The groove 3 is dimensioned to slidably hold a pin 10, as defined below.

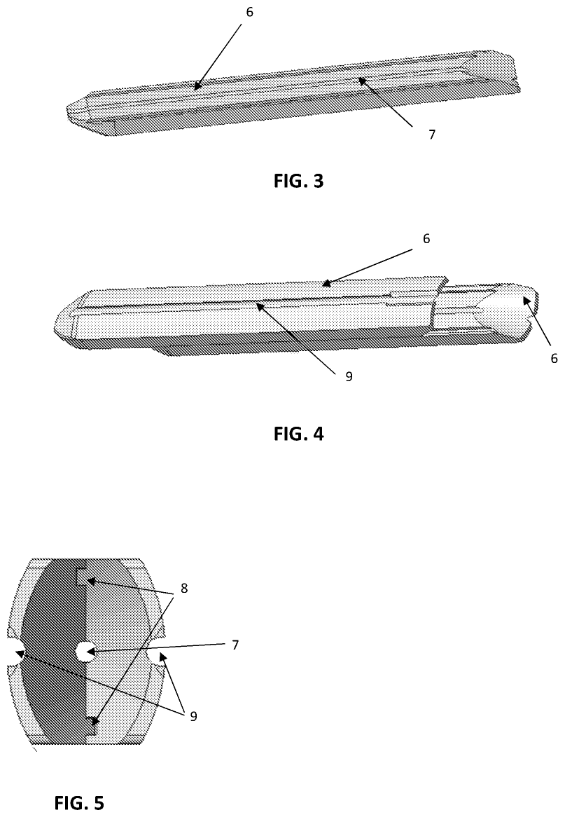

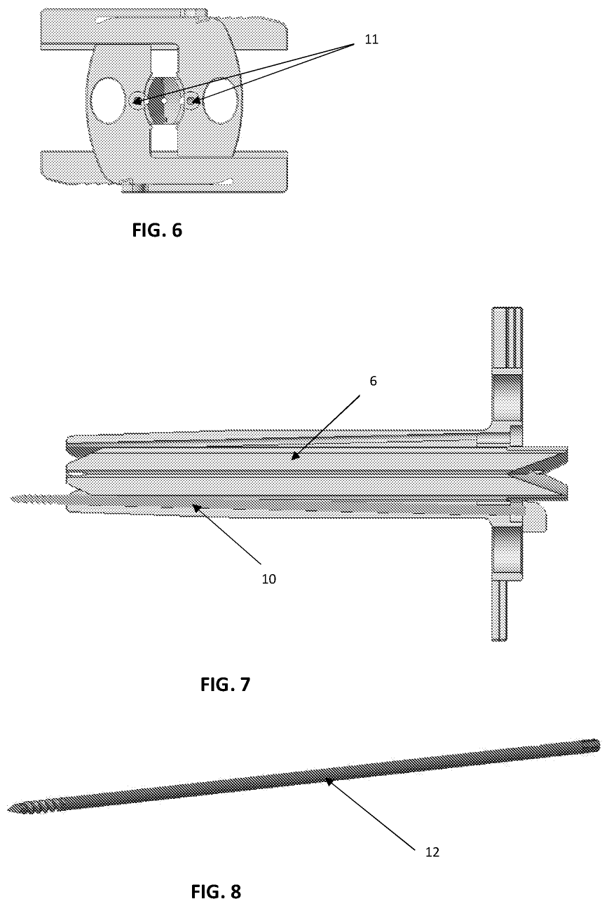

FIG. 3 represents a dilator member 6 and FIG. 4 a dilator that is made by two dilator members 6 that interlock together by means of guides 8.

The dilator is intended to expand the retractor when it is located within the soft tissues.

It has several functions, which includes the generation of an expansion from inside the retractor over the whole length of the blades 1.

The blades 1 are then reinforced and locked in the bone at the bottom with the help of a pin 10 and at the top with the frames 2.

This structure allows to manufacture the blades 1 with a material, such as plastics, that is not mechanically very strong.

As shown on FIG. 4, two dilator members 6 may be brought together to form a progressive dilator, i.e. when introduced between the retractor elements 1,2, the dilator gradually spreads the blades 1 from each other.

On its external face, each dilator member 6 contains a groove 9 which is designed to guide a pin 10. Together with its adjacent blade 1 that also contains a groove 3, a channel 3,9 is created for guiding the pin 10 within the retractor. Such a configuration provides several advantages, in particular an easier and cheaper way to manufacture the grooves (milling or injection of simple surface grooves) than a very long and narrow channel (up 200 mm long Ø3.5 hole) through the whole length of the blade.

The dilator members 6 are also designed to create a K-wire channel 7 for the initial positioning of the retractor system, around the surgical site.

The pin 10 (see FIGS. 7, 8, 9, 13) provides several functions, including to engage the bone, to lock the blades 1 at the bone level in order to prevent the blade flexion under tissues pressure, to reinforce the blades 1 as a metal frame, and it is positioned in an innovative manner that prevents the retractor to go back out of the surgical wound.

The pin 10 can be threaded or not, and can have a driving bit (hexagon, square, male, female,.) at the other end or not. Other shapes can be also contemplated for the pin other end, such as an eyelet, a bent, or an attached plastic part which can be used to retrieve the pin by pulling on it.

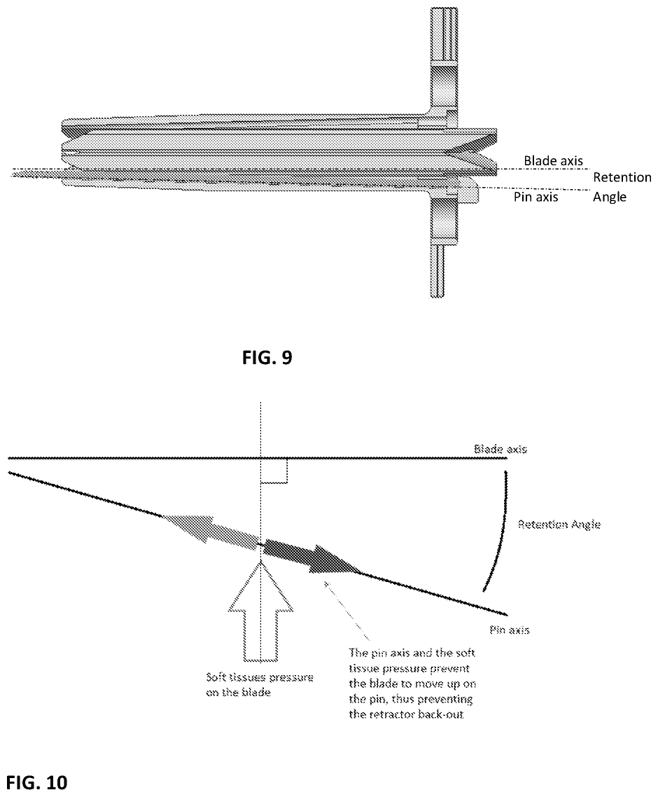

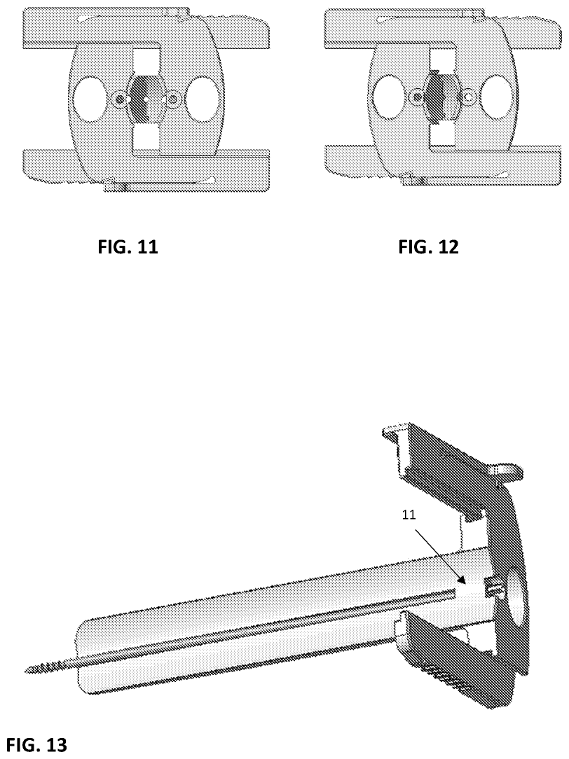

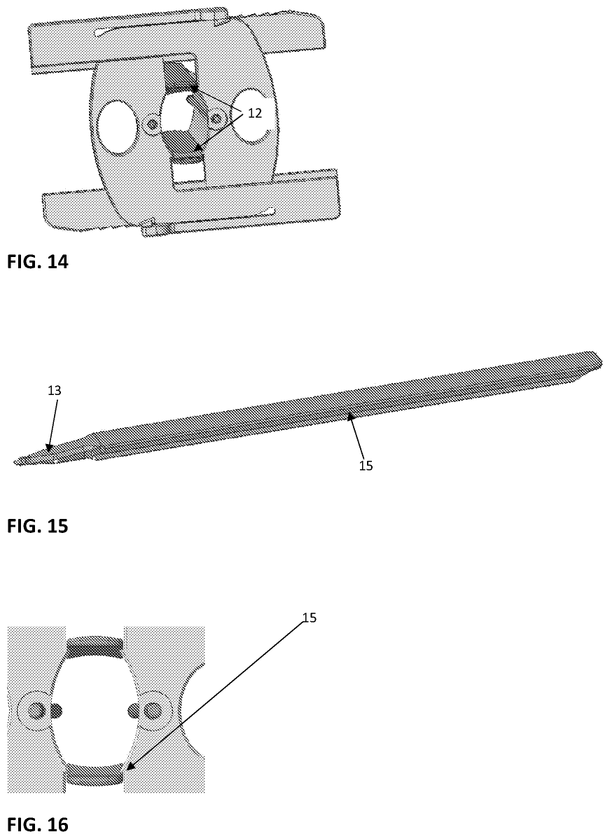

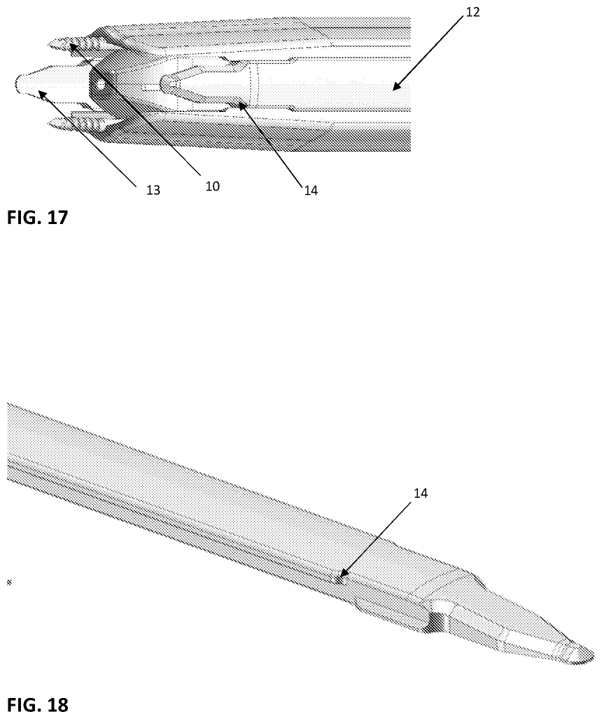

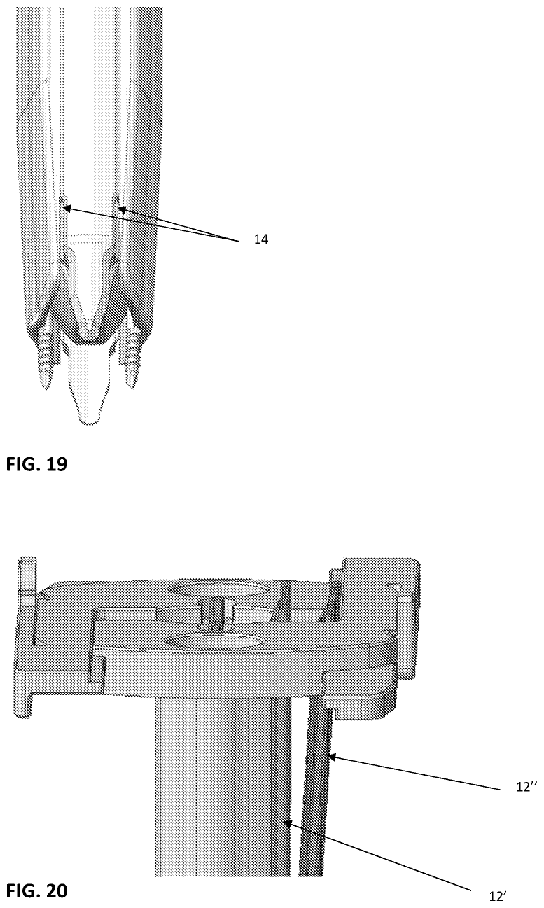

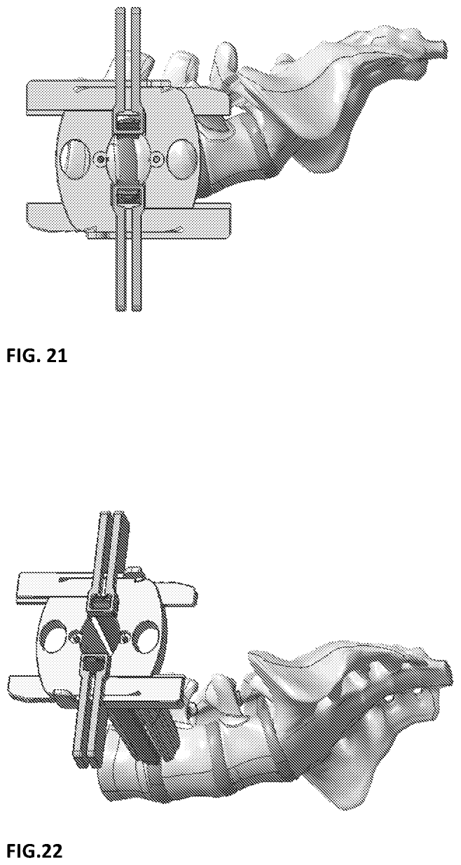

As shown on FIGS. 9 and 10, the channel 3,9 formed by the retractor groove 3 and the dilator groove 9 is oriented along a direction that differs from the blade direction. Consequently, when a pin 10 is located within the channel 3,9, its axis forms an angle, named “retention angle” with respect to the blade axis.

FIG. 10 schematically shows the force (vertical arrow) exerted on the blade inwardly by the surrounding soft tissues. This force has a component (left arrow, gray) that is distally oriented. This additional force prevents an accidental removal of the retractor from the surgical site.

This angle offers a second advantage as it brings the pin entry location a little backward from the internal face of the blade 1, thus making it possible to create a short retention ring 11 that maintains the pin 10 at the frame level even when the dilator is removed.

The shim 12 is a lateral blade that is intended to prevent soft tissues to enter the space between the retractor blades 1.

It has a distal end 13 designed to cut and enter the intervertebral disc, and a groove 15 to guide the shim 12 all along insertion into retractor blades 1 to avoid any deviation toward nerves backwards or vessels at the front of the patient When the shim 12 is well-placed in the disc, linear guiding is interrupted 14 and allows lateral retraction of the shim without removing or expanding the blades 1.

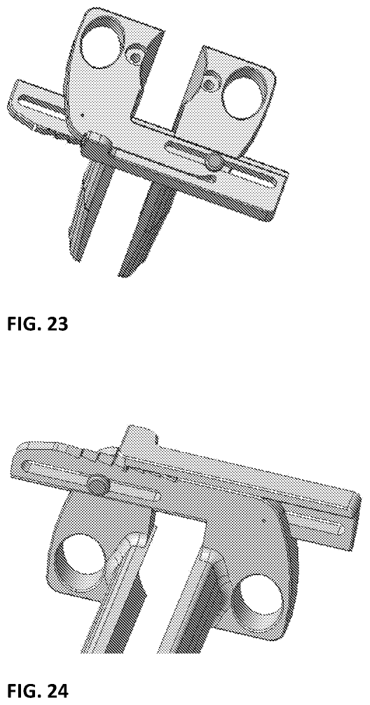

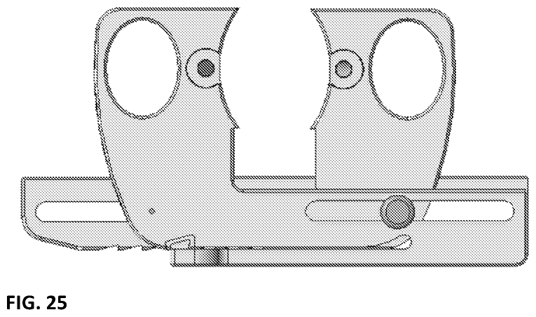

Alternatively, the retractor according to the invention may include only one arm per retractor member. In this case, the frame forms a U-shape. Such an alternative is illustrated on FIGS. 23, 24 and 25. In this example, there is only one ratchet on one side.

Any suitable shape and blocking mechanism for the intended purpose may be used.

The invention is of course not limited to those examples. It includes lateral, anterior and posterior retractor systems.

Same considerations apply to the number of blades and pin per retractor system.

1. Retractor system for spine surgery comprising two retractor members being each made of a blade and a proximal part forming a frame, at least two pins having each a distal end configured to be anchored in a vertebra, and a blocking mechanism for maintaining the retractor members at a certain distance from each other; wherein at least one retractor member contains a channel that is configured to slidably hold a pin; wherein the channel is oriented along a direction that differs from the blade direction.

2. System according to claim 1 wherein the channel is extending along the full blade length.

3. System according claim 1 wherein the channel is a groove.

4. System according to claim 1 wherein the channel distal part is a groove and the channel proximal part is a ring.

5. System according to claim 1 wherein each blade contains a channel that is configured to slidably hold one pin.

6. System according to claim 3 furthermore comprising a dilator for expanding the retractor, wherein said dilator is configured to be slidably mounted between the blades.

7. System according to claim 6 wherein said dilator contains at least one groove that is configured to slidably hold said pin together with the said blade groove

8. System according to claim 6 wherein said dilator is made of two members that interlock together by means of guides

9. System according to claim 6 wherein the dilator has a radio opaque distal end or includes any kind of marker and can be used to measure or control the position of a cage or implant to be placed in between vertebral bodies.

10. System according to claim 1 comprising a shim located between the blades.

11. System according to claim 1 wherein each retractor member includes two arms.

12. System according to claim 1 wherein each retractor member includes only one arm.

13. Retractor system for spine surgery comprising two blades having each a proximal part forming a frame, a blocking mechanism for maintaining the blades at a certain distance from each other, and at least one shim slidably mounted between the blades and comprising lateral linear guiding grooves wherein the guiding grooves end before the shim distal end.

14. Retractor system for spine surgery comprising two blades having each a proximal part forming a frame a blocking mechanism for maintaining the blades at a certain distance from each other, and at least one dilator for expanding the retractor, wherein said dilator is configured to be slidably mounted between the blades

15. System according to claim 14 wherein said dilator contains at least one groove that is configured to slidably hold said pin, together with the said blade groove.

16. System according to claim 14 wherein said dilator is made of two members that interlock together by means of guides.

17. System according to claim 14 wherein the dilator has a radio opaque distal end or includes any kind of marker and can be used to measure or control the position of a cage or implant to be placed in between vertebral bodies.
